(19) United States
(12) Patent Application Publication
(10) Pub. No.: US 2015/0374310 A1
(43) Pub. Date: Dec. 31, 2015

(54) Intelligent Sampling Of Heart Rate

(71) Applicant: Salutron, Inc., Fremont, CA (US)

(72) Inventor: Yong Jin Lee, Palo Alto, CA (US)

(21) Appl. No.: 14/315,680

(22) Filed: Jun. 26, 2014

(51) Int. Cl. A61B 5/00; A61B 5/11; A61B 5/0205

(52) U.S. Cl. CPC A61B 5/7285; A61B 5/0205; A61B 5/1118; A61B 5/4809; A61B 5/024

(57) ABSTRACT

An activity monitor reduces power consumption by providing a heart rate sensor in an inactive mode at times when the heart rate is relatively unimportant. Data from a motion sensor is used to determine when to activate the heart rate monitor to obtain heart rate readings. For example, the motion data may indicate that the user is becoming active after a period of inactivity, or the user is vigorously exercising, then terminates the exercising, or the user is in a sleep-related activity such as the onset of sleep, non-REM (rapid-eye movement sleep) sleep, REM sleep and the user waking from sleep. The heart rate sensor may be in a continuously active mode, an alternating mode, or an inactive mode. In the alternating mode, a delay between readings is set adaptively based on the user's level of activity.

### BACKGROUND

Activity monitors or actigraphs have become popular as a tool for promoting exercise and a healthy lifestyle. An activity monitor can include an accelerometer which can measure motions such as steps taken while walking or running, and estimate an amount of calories used. Moreover, user-specific information such as age, gender, height and weight can be used to tailor the estimate to the user. Such monitors can be worn on the wrist, belt or arm, for instance, or carried in the pocket. The monitor can be worn during an intended workout period or as a general, all day, free living monitor, where the user may perform specific exercises at some times while going about their daily activities at other times, e.g., including sitting, standing and sleeping. An activity monitor can also include a heart rate sensor. There is need to continue the development of such monitors.

### SUMMARY

Devices and techniques are provided herein which reduce power consumption in an activity monitor by limiting the times at which a heart sensor is powered. In one aspect, the heart rate sensor obtains readings according to a schedule, such as once every fifteen minutes, unless there is a reason for obtaining readings more often. On the other hand, a motion sensor, which consumes substantially less power than the heart rate sensor, can obtain readings at a constant rate such as one or more times per second. The heart rate can be useful in determining a calorie burn rate or health-related metrics such as resting heart rate and recovery time after exercise. The motion data can be processed to determines times other than the scheduled times in which it is desirable to obtain heart rate readings either continuously or at a higher rate than a rate which is set by the scheduled times. For example, the motion data may indicate that the user is becoming active after a period of inactivity. Or, the motion data may indicate that the user is vigorously exercising, then terminates the exercising. The heart rate sensor may operate in a continuously active mode when it is desired to obtain heart rate readings at the highest available rate. Or, the heart rate sensor may operate in an alternating mode, where the delay between readings can be set adaptively based on the user's level of activity.

Sleep-related activities of the user may also be detected, such as the onset of sleep, non-REM sleep, REM sleep and the user waking from sleep. REM sleep refers to rapid-eye movement sleep. The heart rate readings can be used to confirm a phase of sleep which is consistent with the motion data.

This summary is provided to introduce a selection of concepts in a simplified form that are further described below in the description. This summary is not intended to identify key features or essential features of the claimed subject matter, nor is it intended to be used to limit the scope of the claimed subject matter.

### BRIEF DESCRIPTION OF THE DRAWINGS

In the drawings, like-numbered elements correspond to one another.

FIG. 1A depicts a front view of an example activity monitor.

FIG. 1B depicts a rear view of the activity monitor of FIG. 1A.

FIG. 2A depicts an example block diagram of circuitry 200 of the activity monitor of FIG. 1A.

FIG. 2B depicts different operating modes of the heart rate sensor mode selection logic 212 of FIG. 2A.

FIG. 2C depicts a process for determining when to sample a user's heart rate.

FIG. 3A depicts a flowchart of an example process used by the heart rate sensor mode selection logic 212 for setting a mode of a heart rate sensor.

FIG. 3B depicts a flowchart of an example process for processing motion data from a motion sensor, consistent with step 300 of FIG. 3A.

FIG. 3C depicts a flowchart of an example process for processing data from a heart rate sensor, consistent with FIG. 3A.

FIG. 3D depicts an interval between active states as a function of an amount of motion and a heart rate while a heart rate sensor is in the alternating mode, consistent with step 321 of FIG. 3A.

FIG. 3E depicts a flowchart of an example process for detecting sleep-related activity in step 341 of FIG. 3A.

FIG. 4A depicts a relationship between activity type and calorie burn rate (CBR) consistent with step 303 of FIG. 3B.

FIG. 4B depicts a relationship between heart rate and calorie burn rate (CBR) consistent with step 303 of FIG. 3A.

FIG. 4C depicts example accelerometer readings of a motion sensor during an activity, consistent with step 301 of FIG. 3B.

FIG. 5A depicts a plot of a calorie burn rate versus time, consistent with FIGS. 5B, 5C and 5D.

FIG. 5B depicts a plot of a heart rate, consistent with FIGS. 5A, 5C and 5D.

FIG. 5C depicts a plot of an amount of motion, consistent with FIGS. 5A, 5B and 5D.

FIG. 5D depicts a plot of a state of a heart rate sensor, consistent with FIGS. 5A, 5B and 5C.

FIG. 6A depicts a time-domain signal from a heart rate sensor during an active state, consistent with step 350 of FIG. 3C.

FIG. 6B depicts heart rate readings consistent with FIG. 3A.

FIG. 6C depicts a spectrum of the time-domain signal of FIG. 6A, consistent with step 351 of FIG. 3C.

FIG. 6D depicts a confidence level consistent with FIG. 6C.

FIG. 7A depicts a plot of breathing rate in different predetermined phases of sleep, consistent with step 340 of FIG. 3A.

FIG. 7B depicts a plot of a slope of the breathing rate of FIG. 7A.

FIG. 7C depicts a plot of heart rate in different predetermined phases of sleep, consistent with FIG. 7A.

### DETAILED DESCRIPTION

Devices and techniques are provided herein which reduce power consumption in an activity monitor by limiting the times at which a heart sensor is powered. An activity monitor is a device which is worn by a user, such as on the wrist, and includes circuitry for detecting heart rate and motion and providing information such as energy expenditure, e.g., calories burned.

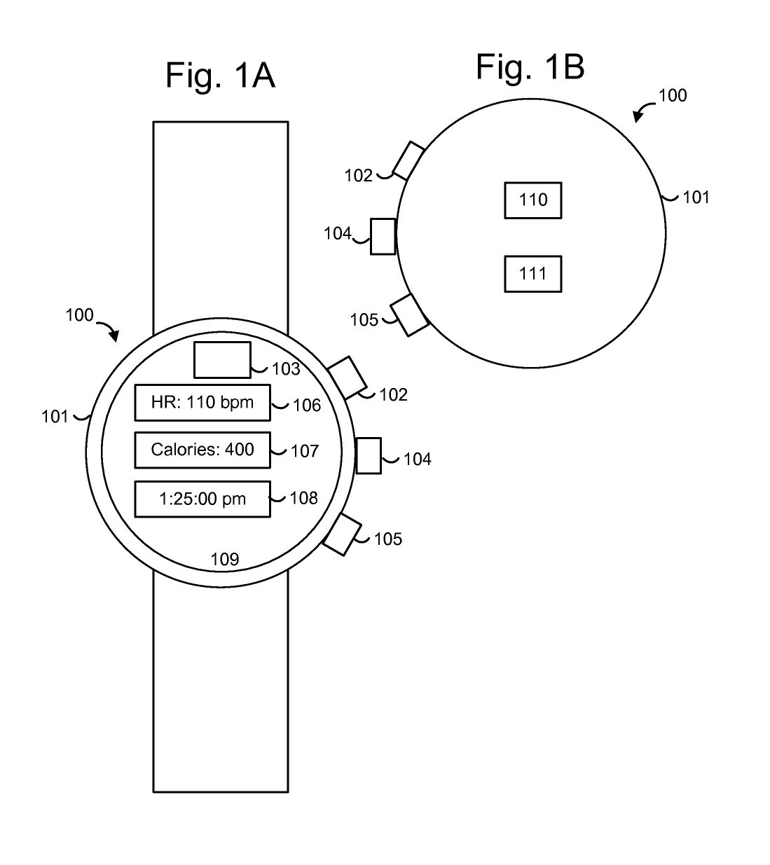

FIG. 1A depicts a front view of an example activity monitor. An activity monitor can be a standalone device which gathers and processes data and displays results to the user. It can be incorporated into another device, or provided as a peripheral of another device, such as a cell phone or other computing device. The activity monitor could also have network connectivity which allows it to communicate its results to a network for further processing and display. For example, the results can be uploaded to a web site via a cell phone, laptop or other networked computing device.

In this example, the activity monitor 100 is a wristwatch type device comprising a watch face and a strap for wearing around the wrist in this example, but other implementations are possible. For example, such monitors can be worn on the belt, head, chest, arm or carried in the pocket. A monitor could also include multiple components which are attached to different parts of the body. For example, the different components can include accelerometers which are attached to different parts of the body, e.g., the arm, leg or foot, to gain a more complete understanding of the user's activity, including posture. The activity monitor 100 includes a case 101, a crown 104, a mode select button 105 and an exercise mode button 102. A display device 109 includes an ambient light sensor 103, a region 106 which depicts a heart rate (HR) (e.g., 110 beats per minutes or bpm), a region 107 which depicts an amount of calories (e.g., 400 calories) consumed in a time period such as in the current day, and a region 108 which depicts a time of day (e.g., 1:25:00 pm). The mode select button 105 may allow the user to activate different operational modes and to input user-specific physiological parameters such as age, gender, height, weight, body mass index or maximum rate of oxygen consumption (VO2max).

The activity monitor can include a heart rate sensor which automatically determines the heart rate continuously, periodically or at other specified times as determined automatically by a control or based on a manual user action. For example, in a free living application, the heart rate can be determined automatically during periods of interest, such as when a significant amount of activity is detected.

The heart rate sensor can use ultrasonic, optical or electrical signals, for instance. For a wrist worn device, it is convenient to use optical transmitters and receivers on the back of the device. These types of monitors are popular since they do not require an electrode-carrying chest strap. In another approach, such an electrode-carrying chest strap can be used in which electrical signals are provided by an ECG-based monitor, where the electrodes of the monitor are constantly in contact with the body and can therefore continuously determine heart rate if desired. Heart rate data can be transmitted from the chest strap to the display device 109 for viewing by the user. The techniques discussed herein are compatible with any type of heart rate monitor.

FIG. 1B depicts a rear view of the activity monitor of FIG. 1A. In this example, an optical component 110 emits light into the user's body and detects reflections, to determine the heart rate. A skin temperature sensor 111 is also provided. Skin temperature can be used to determine activity and calories burned, for instance.

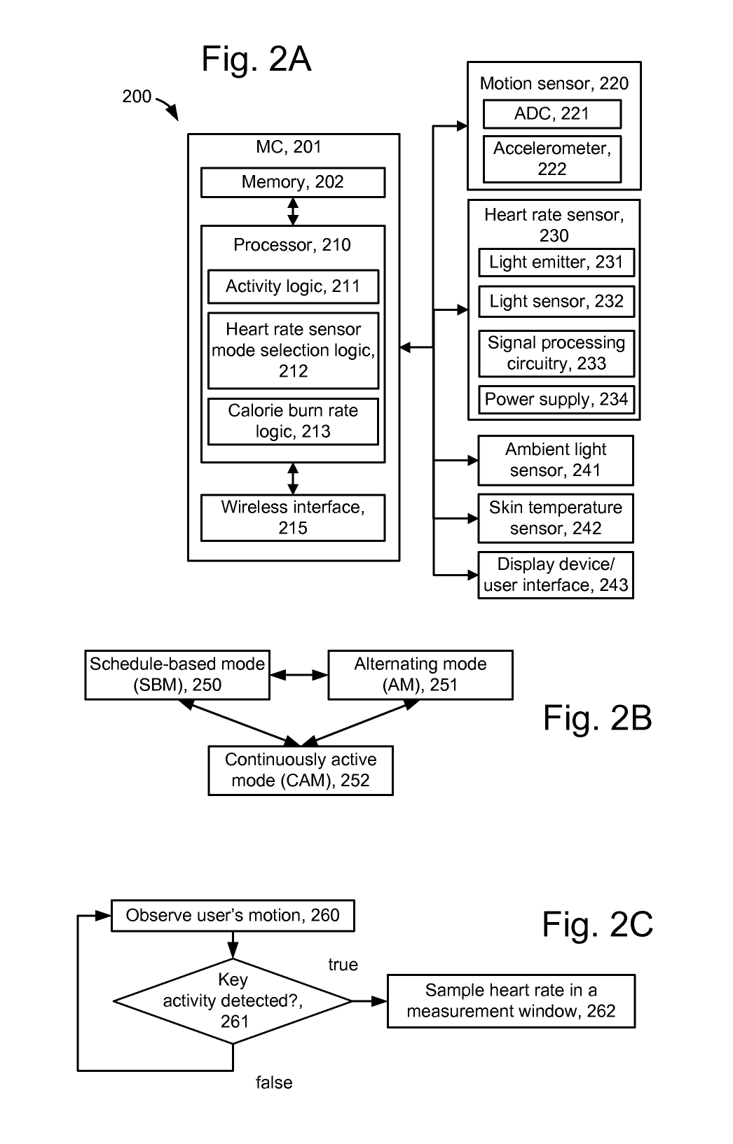

FIG. 2A depicts an example block diagram of circuitry 200 of the activity monitor of FIG. 1A. A micro-controller (microprocessor controller, MC) 201 includes a processor 210 which communicates with a memory 202 and a wireless interface 215. The processor includes activity logic 211 which may determine a type and amount of activity of a user based on information such as motion data from the motion sensor 220, heart rate as determined by the heart rate sensor 230, amount of ambient light as determined by the ambient light sensor 241, skin temperature as determined by the skin temperature sensor 242, and time of day. Heart rate sensor mode selection logic 212 may be used to select a mode such as a schedule-based mode, an alternating mode, and a continuously active mode. Calorie burn rate logic 213 determines a current calorie burn rate and an amount of calories burned over time based on motion and/or heart rate information.

The wireless interface may communicate wirelessly with another computing device such as a cell phone or laptop. For example, the activity monitor may communicate via a piconet such as by using the BLUETOOTH™ protocol.

The micro-controller communicates with a number of components including a motion sensor 220, a heart rate sensor 230, the ambient light sensor 241, the skin temperature sensor 242 and the display device/user interface 243.

The motion sensor 220 includes an accelerometer 222 and an analog-to-digital converter (ADC) 221. The accelerometer may be a three-axis accelerometer. The accelerometer may provide an analog output signal representing acceleration in one or more directions. For example, the accelerometer can provide a measure of acceleration (g-forces) with respect to x, y and z axes. The analog outputs are digitized by the ADC and digital samples (motion data) are provided to the processor 210. Generally, the accelerometer signals can be subject to analog signal processing, analog to digital conversion, time domain processing, conversion to the frequency domain such using a Fast Fourier Transform and frequency domain processing. The ADC could be part of the MC or processor. The accelerometer provides acceleration readings at a prescribed rate such as multiple times per second. The processor can continuously or periodically process samples from the accelerometer. The acceleration samples can be used to determine an activity level of the user and, in some cases, a type of the activity. Based on this, an energy expenditure rate and other metrics can be calculated. Another example of a motion sensor which may be used is a gyrometer, which provides a measure of angular velocity with respect to x, y and z axes. Another example of a motion sensor which may be used is an inclinometer, which provides a measure of pitch, roll and yaw that correspond to rotation angles around x, y and z axes.

In an example implementation, the heart rate sensor 230 includes a light emitter 231, a light sensor 232, signal processing circuitry 233 and a power supply 234. The heart rate sensor determines a current heart rate of a user when it is activated. When activated, power is supplied to the light emitter and other components by the power supply. The signal processing circuitry processes the heart rate signal as discussed further below to obtain heart rate readings. The life of the power supply can be increased by limiting the times at which the heart rate sensor is activated. In one approach, the heart rate senor is provided in an active or inactive state in responsive to a signal from the processor according to the heart rate sensor mode selection logic 212.

The ambient light sensor 241 may include, e.g., a light-dependent resistor or a photodiode and can be used to determine information such as whether the user is in a dark room and therefore is likely sleeping. A light sensor can provide an ambient light reading as a lux value. The The skin temperature sensor 242 may include, e.g., a thermistor, a type of resistor whose resistance varies with temperature, and can be used to determine information such as whether the user is sleeping or exercising. The display device/user interface 243 displays information from the activity monitor and allows the user to enter information such as physiological parameters and to configure settings of the activity monitor. The display device may be used to display information such as a current value of a heart rate, an energy expenditure rate (calorie burn rate) and a cumulative energy expenditure (total calories burned). The user controls may be buttons on the activity monitor which allow the user to enter commands such as to activate the display or configure the activity monitor. The user controls can include the mode select button 105 of FIG. 1A, for instance and associated components.

The diagram is meant to provide a high level understanding of the activity monitor. Specific implementations can take many forms.

The micro-controller may be in communication with each of the other components and transmit signals to them and/or receive signals from them. The memory 202 can store code which is executed by the processor to perform the functionality described herein. This code can include the activity logic, the heart rate sensor mode selection logic and the calorie burn rate logic. The memory is an example of a tangible computer-readable storage apparatus or memory having computer-readable software embodied thereon for programming a processor to perform a method. For example, non-volatile memory can be used. Volatile memory such as a working memory of the processor can also be used. The computer-readable storage apparatus may be non-transitory and exclude a propagating signal.

FIG. 2B depicts different operating modes of the heart rate sensor mode selection logic 212 of FIG. 2A. As mentioned, the heart rate sensor mode selection logic may be used to select a mode such as a schedule-based mode (SBM) 250, an alternating mode (AM) 251 (repeatedly alternating between an active state in which heart rate readings are obtained and an inactive state in which heart rate readings are not obtained), and a continuously active mode (CAM) 252 in which heart rate readings are continuously obtained for as long as the mode is set. Transitions between any two of the modes can occur in specified situations. For example, the schedule-based mode may be implemented when the activity monitor is initially powered on and when motion data indicates that there is no user activity which warrants changing the mode. As the user becomes more active, the alternating mode may be implemented to more closely track the heart rate. As the user becomes even more active, the continuously active mode may be implemented to continuously track the heart rate. For example, continuous tracking may be useful in determining a calorie burn rate, which is highly dependent on heart rate, when the user is very active. The mode can transition from the CAM to the AM and then to the SBM as the user becomes less active.

FIG. 2C depicts a process for determining when to sample a user's heart rate. Step 260 involves observing the user's motion, e.g., based on data from a motion sensor such as an accelerometer. Decision step 261 determines if a key activity is detected. If the decision step is true, step 262 samples the heart rate in a measurement window. If the decision step is false, the observing of the motion continues without sampling the heart rate. The key activity detection can be based on a motion count from the motion sensor, for instance. An activity such as walking or running can be detected based on a signal waveform from the motion sensor. A continuous physical activity can be detected. Periods of limited motion such as the user walking around their home or office can also be detected. The key activity can occur when the motion count is above a threshold while the user is awake. The cessation of physical activity can also trigger heart rate sampling (e.g., to determine a heart rate recover time).

Previous heart rate measurements can also be used to determine whether additional heart rate sampling is warranted. For example, a history of an elevated heart rate or energy expenditure from previous readings may indicate that additional heart rate sampling is warranted. The sampling may continue or be repeated based on the quality, e.g., confidence, of a heart rate reading or a variability in the heart rate readings.

The key activity can also be a sleep-related activity. For example, automated heart rate sampling can occur at the onset of sleep, when the motion counts exceed a threshold during sleep (e.g., indicating REM sleep), or when the user wakes up from sleep (at which time the resting heart rate can be obtained). Generally, when the amount of motion is low, indicating the user is sleeping, automated heart rate variability and respiration rate sampling can be initiated. For example, the heart rate variability can be used to assist in detecting sleep during period of low motion counts. REM sleep detection can occur during periods of moderate motion counts. The user waking up from sleep can also be detected, where the resting heart rate and the variability in the heart rate are also determined. The variability in the heart rate can also be determined during periods of low motion counts while the user is awake. The variability in the heart rate can be used to detect respiratory sinus arrhythmia in which the heart rate varies with the breathing cycle, e.g., how the increases with inspiration and decreases with expiration, in a breathing cycle. These and other features are described in greater detail below.

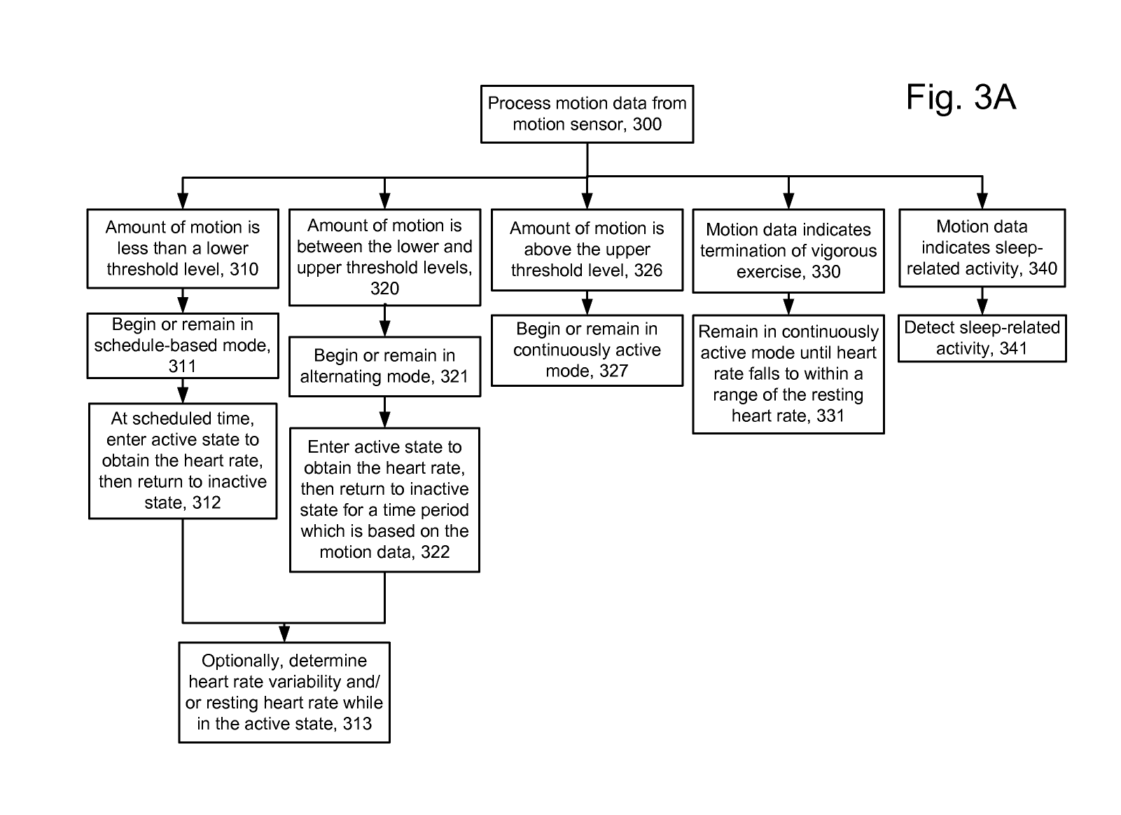

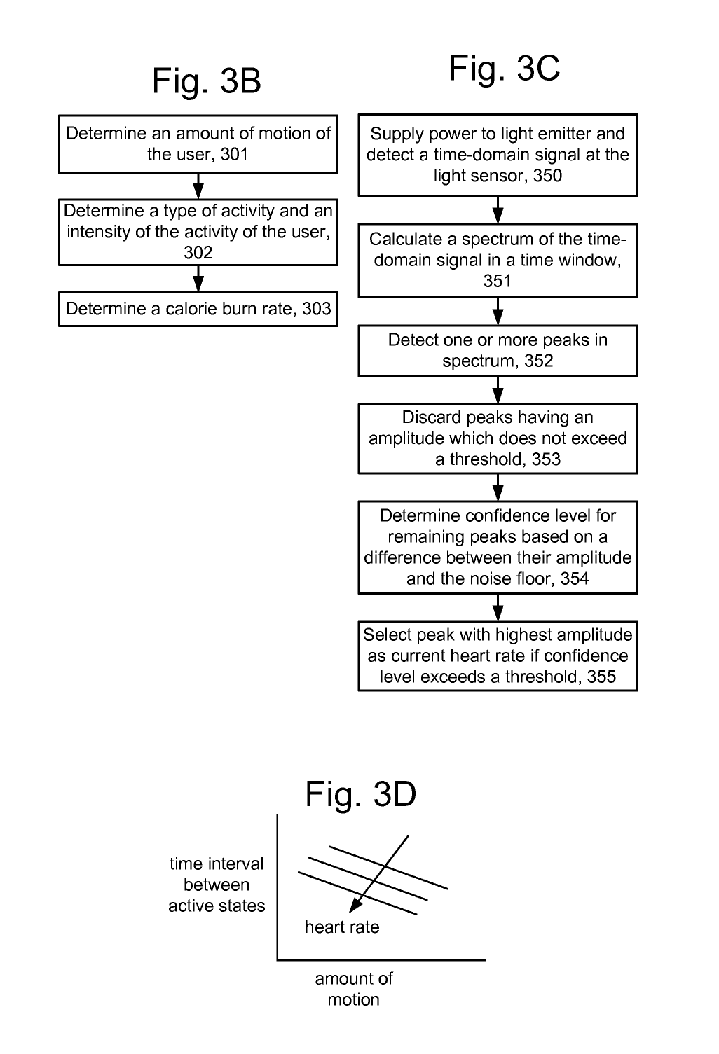

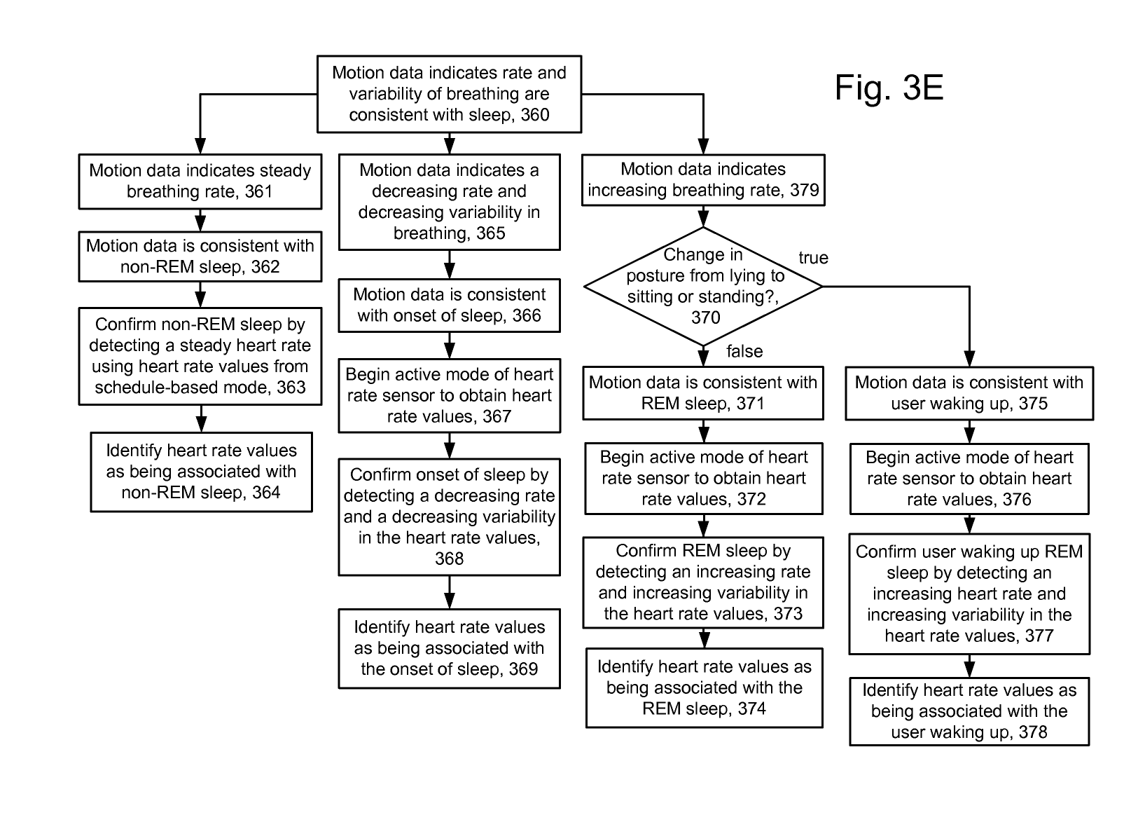

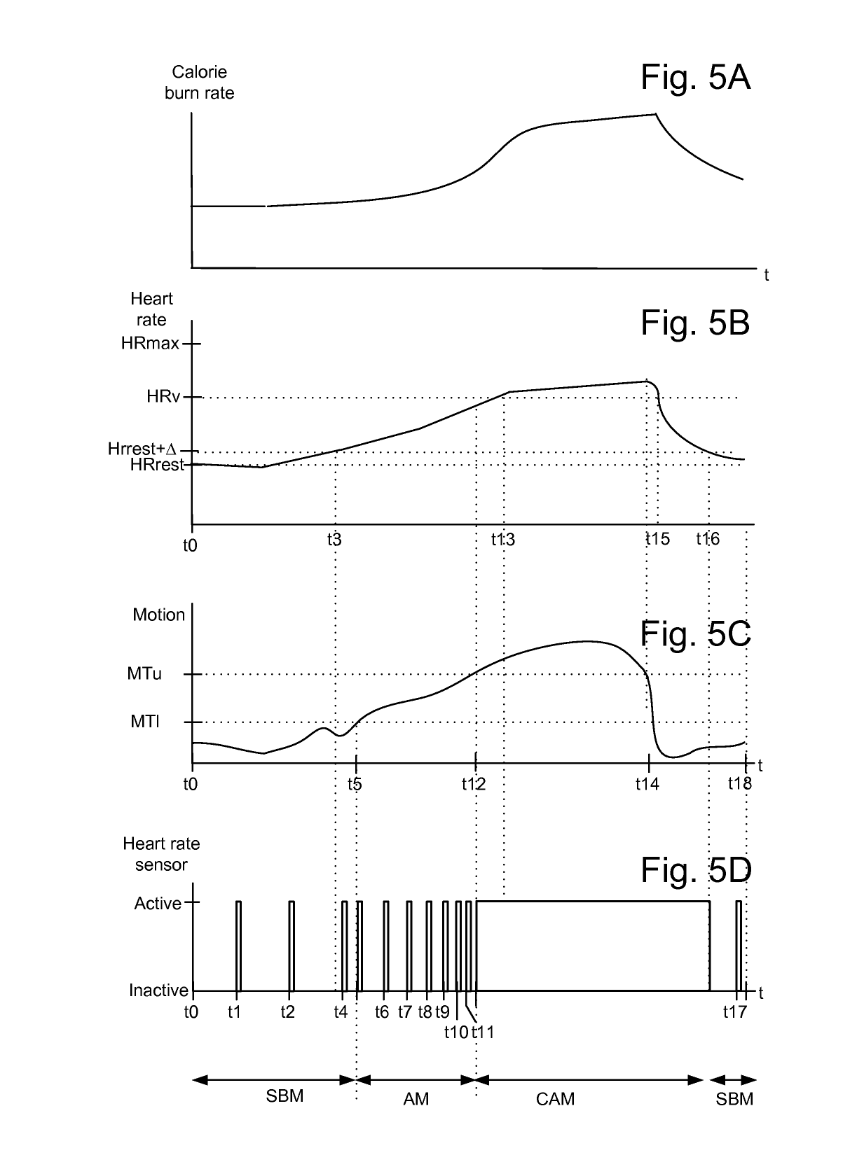

FIG. 3A depicts a flowchart of an example process used by the heart rate sensor mode selection logic 212 for setting a mode of a heart rate sensor. Step 300 involves processing motion data from the motion sensor. For example, the motion data can be continuously provided in response to movements of the user. The motion data can indicate an activity count and a type of activity. Based on the motion data, a number of different paths can be followed. In a first path, at step 310, an amount of motion is less than a lower threshold level of activity (such as MTl in FIG. 5C). That is, the user is inactive and may be involved in an activity such as sitting and reading or watching television. In this case, at step 311, the process involves beginning or remaining in the schedule-based mode of the heart rate sensor. For example, if the amount of motion drops below the lower threshold, the mode can be changed from the alternating mode to the schedule-based mode. If the amount of motion remains below the lower threshold, the mode can remain in the schedule-based mode.

At step 312, at a scheduled time, the active state is entered to obtain the heart rate, then the heart rate sensor returns to the inactive state. In this mode, the heart rate is determined at specified intervals such as every few minutes. This provides a minimal amount of checking on the user's heart rate to save power when there is no indication based on the motion data that more frequent monitoring is desired. Step 313 optionally determines a heart rate variability and/or a resting heart rate while in the active state. The heart rate variability can be used in connection with the sleep-related activity in step 341, for example.

The resting heart rate can be recorded as a metric of the user's health. The resting heart rate is determined while the user is awake but relatively inactive, such as while sitting and reading or watching television.

In a second path, at step 320, an amount of motion is between the lower threshold level and an upper threshold level (such as MTu in FIG. 5C). That is, the user is moderately active but is less than vigorously active and may be involved in an activity such as performing a household chore such as vacuuming In this case, at step 321, the process involves beginning or remaining in the alternating mode of the heart rate sensor. For example, if the amount of motion increases above the lower threshold, the mode can be changed to the alternating mode from the schedule-based mode. If the amount of motion remains between the lower and upper thresholds, the mode can remain in the alternating mode.

At step 322, the active state is entered to obtain the heart rate, then the heart rate monitor returns to the inactive state for a time period. Further, the duration of the inactive state can be fixed or adaptive such as by setting the time period based on the motion data. The duration can be inversely proportional to the amount of motion, such that the duration is relatively shorter when the amount of activity is relatively greater. In one approach, the motion data during the alternating mode comprises an activity count, and the duration of the inactive state is inversely proportional to the activity count. This approach recognizes that it is desirable to check the heart rate more frequently than in the schedule-based mode. However, the amount of activity is not great enough to warrant use of the continuous mode. Thus, some power savings is achieved while still regularly tracking the user's heart rate.

The active state can have a duration which is long enough to obtain a heart rate reading with a desired level of confidence. Generally, this duration will extend over multiple heart beat periods such as several seconds and up to perhaps a minute. The transition to the inactive state can occur when the heart rate reading has been successfully obtained in the active state or when the active state has reached a maximum allowable duration and has thus timed out. That is, the transition to the inactive state can be triggered in response to a determination that the heart rate reading has been successfully obtained in the active state. This allows the active state to continue for as long as is needed, but no longer than is needed, to successfully obtain the heart rate reading.

The active state of the alternating mode differs from the continuously active mode in that the active state has a minimal duration and ends after it successfully obtains a heart rate reading while the continuously active mode will continue to successfully obtain heart rate readings until the mode ends, typically in response to motion data. In one approach, the active state of the alternating mode does not end in response to motion data. Step 313, discussed previously, can also be implemented.

In a third path, at step 326, the amount of motion is above the upper threshold (such as MTu in FIG. 5C). That is, the user is vigorously active and may be involved in an activity such as jogging, bicycling or other vigorous exercising. In this case, at step 327, the process involves beginning or remaining in the continuously active mode. For example, if the amount of motion increases above the upper threshold, the mode can be changed to the continuously active mode from the alternating mode. If the amount of motion remains above the upper threshold, the mode can remain in the continuously active. This approach recognizes that it is desirable to check the heart rate continuously such as to obtain an accurate calorie burn rate during periods of vigorous exercise.

In one approach, the alternating mode and the continuously active mode obtain heart rate readings by detecting each heart beat of the user. Alternatively, there may be cases where readings can be skipped. For example, when there is very little motion when sleeping, a heart rate measurement may be skipped. Thus, in these motion-based sampling modes, we can both add additional sampling points or remove sampling points.

In a fourth path, at step 330, the amount of motion indicates termination of vigorous exercise. That is, the user is suddenly stops the vigorous exercising. For example, the user may be jogging for an extended period of time and then come to a stop. In this case, at step 331, the process involves remaining in the continuously active mode until the heart rate falls to within a range of the resting heart rate. This approach recognizes that it is desirable to determine the heart rate recovery time of the user as a health metric when the user terminates vigorous exercise. The continuously active mode can be ended after an amount of time which is based on the heart rate recovery time, so that power savings are achieved compared to the case where the heart rate sensor remains active after the heart rate recovery time. For example, assuming the resting heart rate (HRrest) is known, the continuously active mode can be ended when the heart rate falls below HRrest+delta in FIG. 5B. The schedule-based mode or the alternating mode can be used after the continuously active mode ends.

In a fifth path, at step 340, the amount of motion indicates a sleep-related activity such as onset of sleep, non-REM sleep, REM sleep and the user waking from sleep. In this case, at step 341, the process involves detecting the sleep-related activity. At this time, one or more of the heart rate sensor modes can be set. This approach recognizes that it is desirable to detect sleep-related activities such as to measure a quality of the user's sleep, e.g., based on a time spent in different phases of sleep. Sleep-related activities can also be indicators of sleep disorders such as snoring, sleep apnea, insomnia, sleep deprivation, and restless legs syndrome. Further details are provided in connection with FIG. 3E.

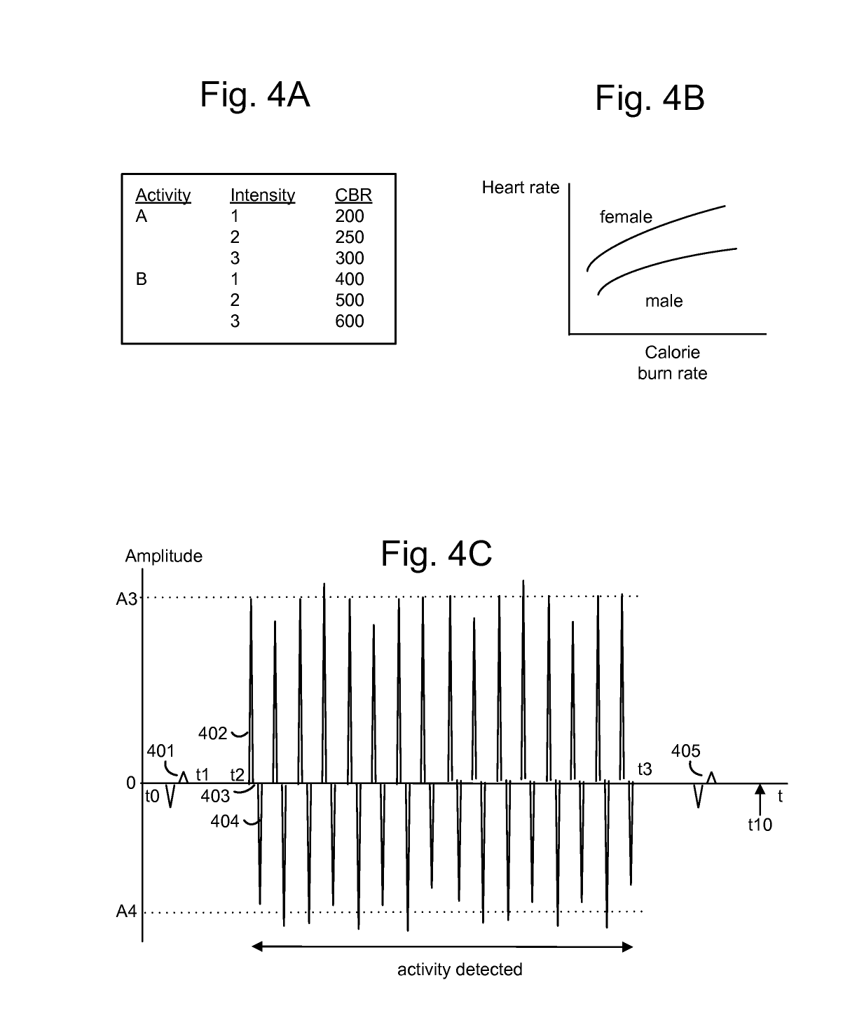

FIG. 3B depicts a flowchart of an example process for processing motion data from a motion sensor, consistent with step 300 of FIG. 3A. Step 310 includes determining an amount of motion of the user. For example, this can be based on an activity count in a time period such as the past few seconds. Step 302 includes determining a type of activity and an intensity of the activity of the user. See FIGS. 4A and 4C for further details. Step 303 includes determining a calorie burn rate. See FIG. 4A for further details. The calorie burn rate can be based on the motion data and/or the heart rate data. For example, the calorie burn rate (CBR) can involve determining a type of activity the user is performing such as by identifying a signature of an activity and an intensity of the activity. In another example, various studies provide a correspondence between heart rate and energy expenditure rate. One study provides an energy expenditure rate as a function of gender, age and weight. For women, the equation is: CBR=−20.4022+0.4472×heart rate−0.1263×weight +0.074×age. For men, the equation is: CBR=−55.0969+0.6309×heart rate+0.1988×weight+0.2017×age. The CBR above is in units of kJ/min, where 1 food calorie=4.2 kJ.

Generally, the motion sensor can detect different predetermined activities. One approach involves identifying a signature of a specific exercise. For example, in a test process, a motion sensor can be worn by a population of users who perform specific exercises and the corresponding accelerometer readings are recorded. This could be done as part of the development of the activity monitor by the manufacturer. The population can represent users with different physiological parameters. A given exercise can be performed with different levels of intensity as well. For example, for running, the intensity can be based on the speed of the user. The speed can be determined from the step rate and an estimated stride, where the stride can be based on factors such as the user's height. Subsequently, when the end user performs a given exercise, the exercise is identified according to a signature based on the physiological parameters of the end user. In another approach, the activity monitor is set up by the end user to recognize particular types of activities as performed by the user in a setup process.

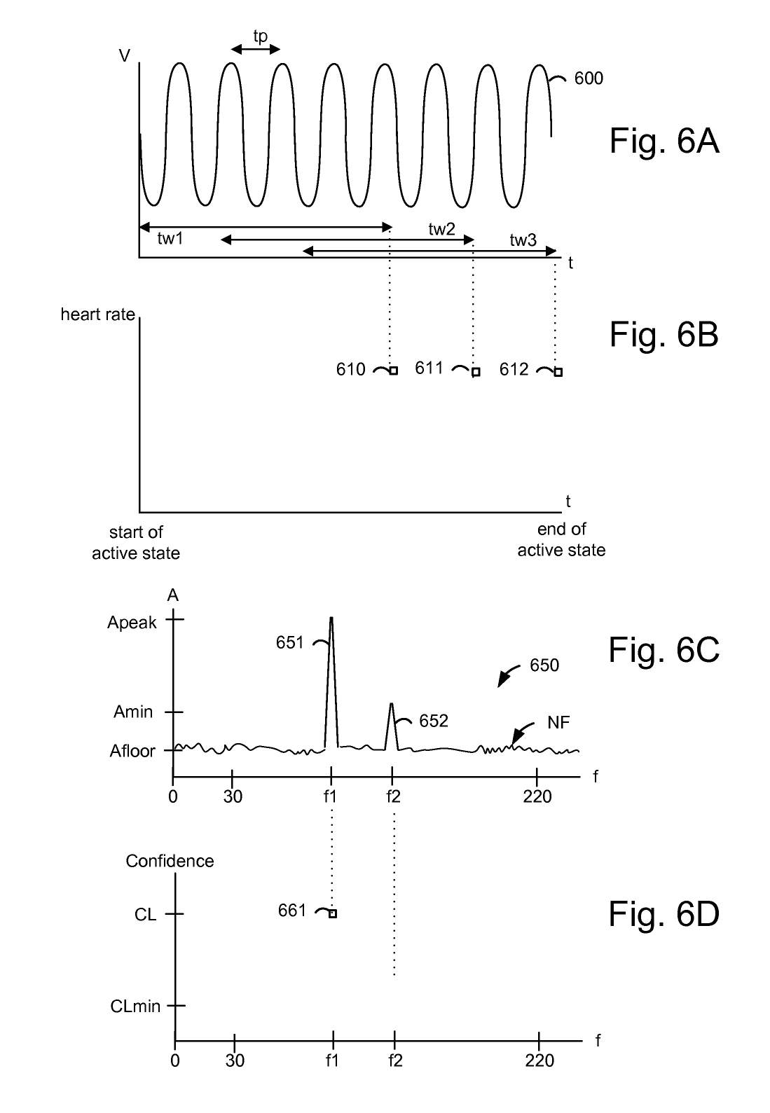

FIG. 3C depicts a flowchart of an example process for processing data from a heart rate sensor, consistent with FIG. 3A. Step 350 involves supplying power to a light emitter and detecting a time-domain signal at the light sensor. The time-domain signal may extend over a time window, such as the past few seconds. See, e.g., FIG. 6A for further details. Step 351 involves calculating a spectrum (frequency-domain signal) of the time-domain signal in the time window such as by using the Fast Fourier Transform (FFT). See, e.g., FIG. 6C for further details. Step 352 involves detecting one or more peaks in the spectrum. Step 353 involves discarding peaks having an amplitude which does not exceed a threshold. See, e.g., FIG. 6C for further details. Step 354 involves determining a confidence level for remaining peaks based on a difference between their amplitude and the noise floor. Step 355 involves selecting a peak with the highest amplitude as the current heart rate if the confidence level exceeds a threshold. This approach provides a heart rate which is most probable and which has at least a minimum confidence level. If the heart rate with the minimum confidence level cannot be determined for a given time window, no heart rate may be output for the time window. Instead, the most recent heart rate with the minimum confidence level can be output.

FIG. 3D depicts an interval between active states as a function of an amount of motion and a heart rate while a heart rate sensor is in the alternating mode, consistent with step 321 of FIG. 3A. In the plot, the horizontal axis depicts an amount of motion and the vertical axis depicts a time interval between active states. The time interval between active states can be inversely proportional to the amount of motion. Thus, a relatively shorter interval can be provided when the amount of motion is relatively greater. This approach recognizes that it is relatively more important to obtain the heart rate when the amount of motion is relatively greater. Further, for a given amount of motion, the time interval between active states can be inversely proportional to the heart rate. Thus, a relatively shorter time interval can be provided when the heart rate is relatively greater. This approach recognizes that it is relatively more important to obtain the heart rate when the heart rate is relatively greater.

Thus, the interval can increase as the user becomes less active and/or has a lower heart rate and increase as the user becomes more active and/or has a higher heart rate. In some cases, the heart rate can be relatively high when the amount of motion is relatively low, such as when the user is performing isometric exercises or weight lifting. Or, the user may by jogging and suddenly stop for a few moments, such as when waiting to cross a street, in which case the heart rate remains high while the motion is low. In other cases, the heart rate can be relatively low when the amount of motion is relatively high, such as when the user is swinging their arms freely while standing still. By accounting for both the amount of motion and the heart rate in setting the delay between active states, power savings can be optimized while increasing the probability that heart rate readings are obtained at times which are useful in determining calorie burn rate and health metrics.

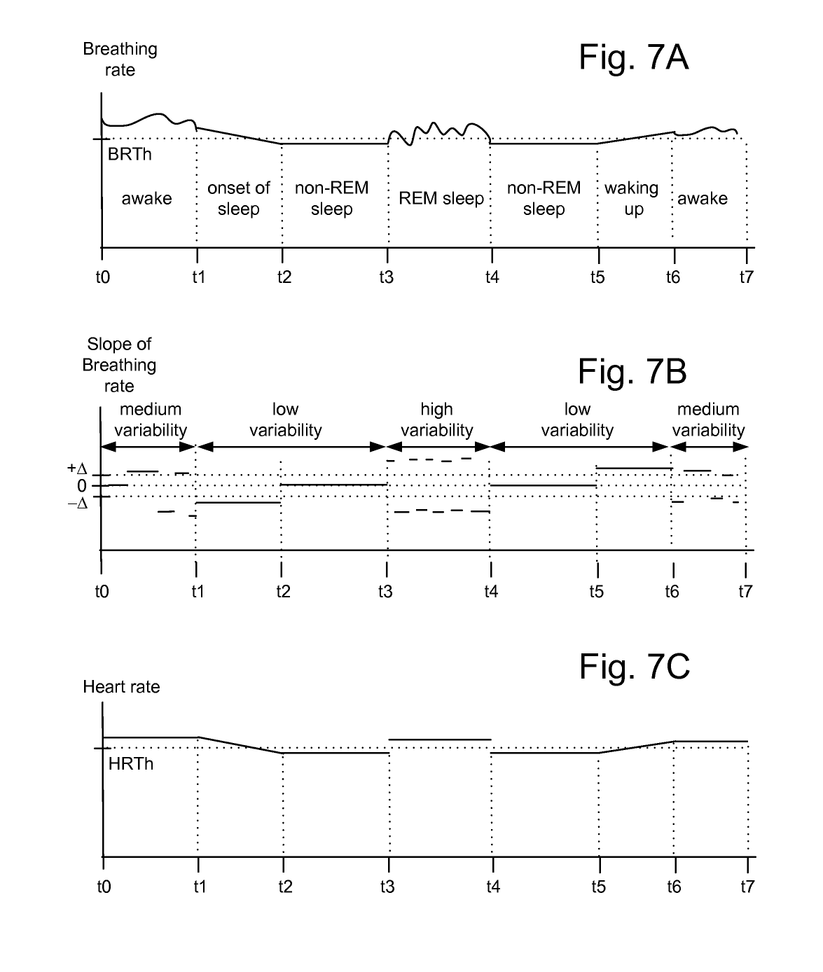

FIG. 3E depicts a flowchart of an example process for detecting sleep-related activity in step 341 of FIG. 3A. See also FIGS. 7A-7C. At step 360, the motion data indicates that a rate and variability of breathing are consistent with sleep. In general, physiological functions such as brain wave activity, breathing, and heart rate have a high variability when a person is awake or during REM sleep, an active type of sleep. On the other hand, these functions have a low variability when a person is in non-REM sleep, an inactive type of sleep. For instance, when a person is awake, the breathing rate can be variable since it is affected by speech, emotions, exercise, posture, and other factors. With the onset of sleep, as a person progress from wakefulness to non-REM sleep, the breathing rate slightly decreases and becomes very regular. The heart rate also decreases and has decreased variability. During REM sleep, the pattern becomes much more variable again, with an overall increase in breathing rate similar to the wakeful state. The heart rate also increases and has increased variability. The motion sensor can detect the breathing of the user such as when the motion monitor is in a position such that it will move when the user inhales and exhales. For example, a wrist worn activity monitor will move when the user has their hand on their torso and the torso moves due to inhaling and exhaling, or when movement of the torso is translated to movement of the arm and wrist.

In a first approach, at step 361, the motion data indicates a steady breathing rate. At step 362, the motion data is consistent with non-REM sleep. Step 363 confirms that the user is in non-REM sleep by detecting a steady heart rate using heart rate values from the heart rate sensor. In one approach, the heart rate sensor can remain in the schedule-based mode for maximum power savings. For example, heart rate values of 60, 60, and 60 bpm may be obtained at scheduled times such as at 10 minute intervals. This is consistent with the time period of t2-t3 and t4-t5 in FIG. 7A. Step 364 identifies the heart rate values as being associated with non-REM sleep. The time in which the user is in the non-REM sleep, and the associated heart rate values, can be logged for reporting to the user or for further analysis. The heart rate sensor could alternatively be in the alternating mode or the continuously active mode.

In a second approach, at step 365, the motion data indicates a decreasing rate and a decreasing variability in the breathing rate. At step 366, the motion data is consistent with the onset of sleep. Step 367 begins the active mode of the heart rate sensor to obtain heart rate values. Step 368 confirms the onset of sleep by detecting a decreasing rate and a decreasing variability in the heart rate values. In this case, since the user's physiology is changing, it is desirable to obtain a current heart rate value rather than rely on an older heart rate value which may have been obtained in the schedule-based mode. In some cases, it is sufficient to enter the active mode to obtain a heart rate reading and then return to the schedule-based mode. Power is saved compared to the case of continuously obtaining heart rate readings while the user sleeps. For example, heart rate values of 62, 61, and 60 bpm may be obtained in three successive active states, which could be several seconds or minutes apart. This is consistent with the time period of t1-t2 in FIG. 7A. Step 369 identifies the heart rate values as being associated with the onset of sleep. The time in which the user is in the onset of sleep, and the associated heart rate values, can be logged for reporting to the user or for further analysis.

In a third approach, at step 379, the motion data indicates an increasing breathing rate. Decision step 370 determines if there is a change in the user's posture from lying to sitting or standing. For example, the orientation of the activity monitor can indicate the posture. Lying is associated with the arm being generally horizontal. A transition from lying to sitting is associated with a substantial arm movement such as swinging the arm. Standing is associated with the arm being generally horizontal.

If decision step 370 is false, step 371 determines that the motion data is consistent with REM sleep. Step 372 begins the active mode of the heart rate sensor to obtain heart rate values. Step 373 confirms the REM sleep by detecting an increasing rate and an increasing variability in the heart rate values. In this case, since the user's physiology is changing, it is desirable to obtain a current heart rate value rather than rely on an older heart rate value which may have been obtained in the schedule-based mode. For example, heart rate values of 60, 61, and 62 bpm may be obtained in three successive active states. This is consistent with the time period of t3-t4 in FIG. 7A. Step 374 identifies the heart rate values as being associated with REM sleep. The time in which the user is in the REM sleep, and the associated heart rate values, can be logged for reporting to the user or for further analysis.

A further confirmation that the user is in REM sleep can involve detecting a subsequent transition to the non-REM sleep which is indicated by a decreasing rate and a decreasing variability in the breathing rate and/or heart rate values. A further confirmation that the user is in REM sleep is based on the user being in non-REM sleep directly before the REM sleep is detected.

If decision step 370 is true, step 375 determines that the motion data is consistent with the user waking up. Step 376 begins the active mode of the heart rate sensor to obtain heart rate values. Step 377 confirms that the user is waking up by detecting an increasing rate and an increasing variability in the heart rate values. In this case, since the user's physiology is changing, it is desirable to obtain a current heart rate value rather than rely on an older heart rate value which may have been obtained in the schedule-based mode. Step 378 identifies the heart rate values as being associated with the user waking up. For example, heart rate values of 60, 61, and 62 bpm may be obtained in three successive active states. This is consistent with the time period of t5-t6 in FIG. 7A. The time in which the user is waking up, and the associated heart rate values, can be logged for reporting to the user or for further analysis. A resting heart rate can also be determined at this time, e.g., by selecting one of the heart rates from the successive active states, such as a lowest heart rate among them. In another approach, the resting heart rate is determined from an average or median of the heart rates.

FIG. 4A depicts a relationship between activity type and calorie burn rate (CBR) consistent with step 303 of FIG. 3B. In a simplified example, different activities, e.g., Activity 1 or 2, and different intensities, e.g., 1, 2 and 3 can be associated with calorie burn rates (CBR). Calorie burn rates can be provided for repetitive activities such as certain exercises and non-repetitive activities such as sleeping and sitting. For instance, the various activities can include: cycling, calisthenics, weight lifting, rowing, aerobics, stretching, dancing, running, bowling, golf, jumping rope, skateboarding, playing tennis, swimming, gardening, cleaning, and so forth. Other activities which are not necessarily exercises can similarly be detected such as sleeping, sitting, talking and so forth. A calorie burn rate can be associated with each activity and intensity level. The calorie burn rate can be adjusted based on physiological parameters of the user as well.

Generally, an energy expenditure, in terms of food calories (kcal) per minute (a calorie burn rate or CBR), can be associated with each activity and intensity. Moreover, the energy expenditure rate can be adjusted based on the user's physiological parameters. For a given activity, the energy expenditure rate is higher when the intensity is higher. Also, the energy expenditure rate is higher when the user's weight is higher. The energy expenditure rate is strongly dependent on weight. For example, for the activity of running at 5 mph, the energy expenditure rate is 472, 563, 654 or 745 calories per hour based on a user weight of 130, 155, 180 or 205 pounds, respectively. For the same activity but with a higher intensity of running at 6 mph, the energy expenditure rate is 590, 704, 817 or 931 calories per hour based on a user weight of 130, 155, 180 or 205 pounds, respectively. In some cases, the user is resting, such that a basal metabolic rate (BMR) or a resting metabolic rate (RMR) applies. The BMR applies to a user who has just awoke after sleeping while the RMR applies when the user is awake but resting. BMR and RMR are a function of weight, height and age.

FIG. 4B depicts a relationship between heart rate and calorie burn rate (CBR) consistent with step 303 of FIG. 3A. The graph depicts calorie burn rate on the horizontal axis and heart rate on the vertical axis. This relationship depends on physiological parameters such as gender. Typically, a male has a higher CBR than a female for a given heart rate. Other factors such as age, weight and physical condition are also relevant. Generally, the CBR increases non-linearly with heart rate. The CBR increases at an increasingly higher rate as heart rate increases. The techniques described herein recognize when heart rate values are useful in accurately calculating CBR without continuously monitoring the heart rate.

FIG. 4C depicts example accelerometer readings of a motion sensor during an activity, consistent with step 301 of FIG. 3B. The plot depicts a relatively high amount of activity, where a repetitive pattern is detected. An accelerometer has the ability to measure acceleration in one, two or three directions, such as along the x, y and z axes of a Cartesian coordinate system. The magnitude of acceleration can be determined as well. In some cases, the acceleration is not recorded unless it exceeds threshold. A movement of a user is represented by acceleration readings, e.g., along the x, y and z axes. In one approach, each movement results in an activity count. Thus, the motion data comprises an activity count and the motion data is consistent with the user engaging in the vigorous exercising when the activity count exceeds a threshold count in a time period, e.g., twenty counts per minute. Each count could correspond to a motion during exercise such as a foot step during jogging.

Generally, the intensity level of activity of a user over time can be determined based on the acceleration readings. For example, amplitude, frequency and zero-crossings of the acceleration can be used to determine a level of the activity. Higher amplitudes, frequencies and zero-crossings are associated with a higher activity level.

In this example, time extends on the horizontal axis and amplitude is on the vertical axis. The amplitude is from a motion sensor such as one or more accelerometers. The amplitude could represent a component (Ax, Ay, Az) along one of the x, y and z axes of an amplitude vector. Or, the amplitude could represent the magnitude of an amplitude vector, e.g., the square root of Ax̂2+Aŷ2+Aẑ3. The amplitude extends generally between A4 and A3. Acceleration readings 401 and 405 indicate small movements. In contrast, acceleration readings such as 402 and 404, with a zero crossing 403 between them, indicate larger, relatively high frequency movements. For example, the user may be running The larger, relatively high frequency movements extend from t2-t3.

In some cases, the type of exercise that a user is performing can be detected based on characteristics of the accelerometer readings. For example, a training process may be performed in one or more users perform specified exercises and the resulting accelerometer readings are recorded. Accelerometer readings from a subsequent exercise period can be compared to the recorded accelerometer readings (signatures) to identify the exercise being performed, as well as a pace of the exercise based on the frequency of movement. For example, it may be determined that a user is running at 3 miles per hour. The type of exercise which is performed and the pace of the exercise can further be correlated with a rate of calories burned by the user based on scientific studies which have been published. The rate of calories burned can be tailored to a particular user based on physiological factors such as age, gender, height and weight. This information can all be encompassed within the activity logic 211 of the processor 210 (FIG. 2A) using appropriate formulas and tables.

If the type of activity cannot be detected, a general level of activity of the user can be detected (e.g., little motion, moderate motion, high motion) and a CBR associated with that level. In some cases, additional sensors such as GPS can be used to determine the current activity of a user. For example, GPS can be used to determine the speed of movement of a user.

FIG. 5A depicts a plot of a calorie burn rate versus time, consistent with FIG. 5B, 5C and 5D. The horizontal axis depicts time and the vertical axis depicts calorie burn rate (CBR). In this example, the CBR increases as a person begin vigorously exercising and subsequently decreases. The horizontal axes in FIGS. 5A-5D are time aligned.

FIG. 5B depicts a plot of a heart rate, consistent with FIGS. 5A, 5C and 5D. The horizontal axis depicts time and the vertical axis depicts heart rate. HRv is a threshold for detection of vigorous exercise. HRrest is the resting heart rate. HRrest+delta represents a range above HRrest. The heart rate increases above HRrest at t3 until it exceeds HRv at t13. The heart rate subsequently decreases below HRv at t15. Delta represents a range above the resting heart rate of, e.g., 5 beats per minutes. HRv is above the range of the resting heart rate (HRrest+delta) by at least a specified amount, e.g., 30-60 beats per minute.

FIG. 5C depicts a plot of an amount of motion, consistent with FIGS. 5A, 5B and 5D. The horizontal axis depicts time and the vertical axis depicts motion. MTv is a threshold for detection of vigorous exercise. MTu is an upper threshold, e.g., for detection of vigorous exercise. MTl is a lower threshold, e.g., for detection of non-vigorous activity. The amount of motion increases above MTl at t5 until it exceeds MTu at t12. The amount of motion subsequently decreases below MTu at t14.

FIG. 5D depicts a plot of a state of a heart rate sensor, consistent with FIGS. 5A, 5B and 5C. The horizontal axis depicts time and the vertical axis depicts whether the heart rate sensor is in an inactive or active state. In this example, the heart rate sensor is in the schedule-based mode (SBM) from t0-t5, the alternating mode (AM) from t5-t12, the continuously active mode (CAM) from t12-t16 and the SBM from t16-t18. In the SBM from t0-t5, the active state is selected at t1, t2 and t4. In this example, the active state is selected at equal intervals. Note that the time values t0, t1 and so forth and not necessarily equally spaced. As mentioned, the active state may be maintained for a minimum time period which allows a heart rate reading to be obtained with at least a minimum level of confidence. In the alternating mode (AM) from t5-t12, the active state is selected at t5, t6, t7, t8, t9, t10 and t11. Moreover, the interval between the active state becomes progressively smaller in proportion to the amount of motion becoming progressively larger (FIG. 5C). In the CAM from t12-t16, the active state is selected continuously. In the SBM from t16-t18, the active mode is selected at t17. The time intervals from t0-t1, t1-t2, t2-t4 and t16-t17 may be equal in one approach.

Accordingly, it can be seen that a method for monitoring a heart rate of a user comprises: obtaining motion data from a motion sensor worn by the user which indicates the user is not engaging in a threshold level of activity (e.g., from t0-t5 where the motion is less than MTl); keeping a heart rate sensor worn by the user in a schedule-based mode in response to the motion data indicating the user is not engaging in the threshold level of activity, where the heart rate sensor in the schedule-based mode obtains a heart rate of the user at scheduled times which are not based on the motion data and does not obtain a heart rate of the user at other times; obtaining motion data from the motion sensor which indicates the user is engaging in the threshold level of activity (e.g., after t5, where the motion is above MTl); in response to the motion data which indicates the user is engaging in the threshold level of activity, providing the heart rate sensor in an alternating mode instead of in the schedule-based mode (e.g., from t5-t12), where the heart rate sensor in the alternating mode repeatedly alternates between an active state in which the heart rate sensor obtains the heart rate of the user and an inactive state in which the heart rate sensor does not obtain the heart rate of the user; obtaining motion data from the motion sensor during the alternating mode; and setting a duration of the inactive state based on the motion data which is obtained from the motion sensor during the alternating mode. For example, a first duration of the inactive state is t6-t5 less the duration of the active mode which begins at t5, a second duration of the inactive state is t7-t6 less the duration of the active mode which begins at t6, and so forth.

In another aspect, a method for monitoring a heart rate of a user comprises: determining that motion data from a motion sensor worn by a user is not consistent with the user engaging in vigorous exercising in a first time period (e.g., from t5-t12, where the motion is less than MTu); in response to the determining that the motion data in the first time period is not consistent with the user engaging in vigorous exercising, providing a heart rate sensor of the user in an alternating mode in the first time period, where the heart rate sensor in the alternating mode repeatedly alternates between an active state in which the heart rate sensor obtains a heart rate of the user and an inactive state in which the heart rate sensor does not obtain the heart rate of the user; determining that motion data from the motion sensor is consistent with the user engaging in vigorous exercising in a second time period directly after the first time period (e.g., from t12-t14, where the motion is greater than MTu); in response to the determining that the motion data in the second time period is consistent with the user engaging in vigorous exercising, providing the heart rate sensor continuously in the active state in the second period; determining that motion data from the motion sensor is not consistent with the user engaging in the vigorous exercising in a third time period (e.g., from t12-t16, where the motion is less than MTu) directly after the second time period; in response to the determining that the motion data in the third time period is not consistent with the user engaging in vigorous exercising, keeping the heart rate sensor continuously in the active state until the heart rate is determined to have to fallen to within a range of a resting heart rate of the user (e.g., HRrest+delta); and storing, as a heart rate recovery time of the user, a time elapsed between a start of the third time period (e.g., t14), when the motion data in the third time period initially indicates the user has terminated the vigorous exercising, and a time at which the heart rate is determined to have to fallen to within the range of the resting heart rate of the user (e.g., t16).

In another aspect, a monitor comprises: a heart rate sensor (230) worn by a user; a motion sensor (220) worn by the user; and a processor (210). The processor: obtains motion data from the motion sensor which indicates the user is not in a predetermined phase of sleep (e.g., from t0-t1 and t6-t7 due to the breathing rate being above BRTh and the variability being above a threshold); keeps the heart rate sensor in a schedule-based mode in response to the motion data indicating the user is not in the predetermined phase of sleep, the heart rate sensor in the schedule-based mode obtains a heart rate of the user at scheduled times which are not based on the motion data and does not obtain a heart rate of the user at other times; obtains motion data from the motion sensor which indicates the user is in the predetermined phase of sleep (e.g., from t1-t3 or t4-t6 due to the breathing rate being below BRTh and the variability below above a threshold; or from t3-t4 due to the breathing rate being above BRTh and the variability being above a threshold); and in response to the motion data which indicates the user is in the predetermined phase of sleep, provides the heart rate sensor in an active state in which the heart rate sensor obtains values of the heart rate of the user at times which are outside of the scheduled times and identifying the values of the heart rate which are obtained while the heart rate sensor is in the active state as being associated with the predetermined phase of sleep.

FIG. 6A depicts a time-domain signal from a heart rate sensor during an active state, consistent with step 350 of FIG. 3C. The horizontal axis depicts time and the vertical axis depicts voltage. One example of heart rate sensor injects light into a user's body such as from the back of a wrist worn activity monitor, and senses reflections of the light from the body. The amplitude of the reflections can be depicted by a time varying voltage which varies with the movement of blood vessels in the body. This movement can involve the periodic expansion and contraction of blood vessels at the frequency of the heart rate, for instance. In this example, the time-domain signal is plotted using voltage on the vertical axis and time on the horizontal axis. The signal may be generally sinusoidal with periodic peaks. An example period between the peaks is tp. The period, which is the inverse of the heart rate, can vary over time depending on what the user's activity. Although, over a few seconds, the heart rate may be fairly steady.

In calculating the spectrum of the time-domain signal, one approach is to transform a portion or window of the signal. For each new reading, the window is moved and the transform is based on the portion of the signal in the current window. The duration of a window should be sufficient to capture the frequency characteristic by encompassing two or more peaks at the lowest expected heart rate, corresponding to the longest heartbeat period. For example, if the lowest expected heart rate is thirty beats per minute (bpm), corresponding to a period of two seconds, the window should be at least two seconds. In practice, the window can be longer, such as 5-6 seconds, to accurately capture the heart beat period. If the window is too long, the current value of the heart rate will be averaged out with previous values, and the computational cost increases. The spectrum obtained from each window results in a reading of the heart rate. Each window can overlap by, e.g., 1-2 seconds so that a new heart rate value is obtained every 1-2 seconds. Example windows tw1, tw2 and tw3 are depicted.

FIG. 6B depicts heart rate readings consistent with FIG. 3A. The horizontal axis depicts time, aligned with the horizontal axis of FIG. 6A, and the vertical axis depicts heart rate. Readings 610, 611 and 612 are obtained from windows tw1, tw2 and tw3, respectively. The time axis may extend from the start of an active state to the end of the active state, e.g., in a measurement window. As mentioned, the active state can extend for a time which is sufficient to obtain a heart rate reading with at least a minimum level of confidence. In this example, the readings 610 and 611 do not have the minimum level of confidence but the reading 612 does have the minimum level of confidence. As a result, the active state is terminated after the reading 612. Other approaches are possible. For example, the active state may continue until a number n>=1 of successive readings are obtained with the minimum level of confidence, or until a specified percentage of readings, e.g., at least 3 out of 5, are obtained with the minimum level of confidence.

FIG. 6C depicts a spectrum of the time-domain signal of FIG. 6A, consistent with step 351 of FIG. 3C. A spectrum can be obtained for each of the time windows of FIG. 6A, for instance. In this example, the spectrum 650 is obtained for tw3. The spectrum can be obtained using a Discrete Fourier transform (DFT) such as the FFT. The spectrum is plotted using an amplitude on the vertical axis (which may be a logarithmic scale) and frequency (e.g., heart rate) on the horizontal axis. The spectrum can be an amplitude spectrum or power spectrum, for instance. The single-sided power spectrum of a voltage waveform is in units of Volts rms squared. The amplitude spectrum is obtained by taking the square root of the power spectrum. In this example, heart rates between 30 and 220 bpm are considered to be valid for a human. The noise floor (NF) represents the lowest possible amplitude (Afloor) of the spectrum. The theoretical noise floor of the FFT is equal to the theoretical signal to noise ratio plus the FFT process gain, 10×log(M/2), where M is the size of the FFT.

Here, there is a peak 651 at a frequency of f1 with an amplitude of Apeak. F1 is the heart rate of the reading 612. The peak is not discarded because its amplitude is above a minimum threshold of Amin, consistent with step 353 of FIG. 3C. There is another peak 652 at a frequency of f2. However, its amplitude is less than Amin so it is discarded. This peak is present due to noise. A confidence level of the peak 651 can be determined as CL=Apeak−Afloor (e.g., decibels or Volts rms squared), for instance, consistent with step 354 of FIG. 3C, or some other function proportional to the amount by which Apeak exceeds Afloor.

FIG. 6D depicts a confidence level consistent with FIG. 6C. The horizontal axis depicts frequency, aligned with the horizontal axis of FIG. 6C, and the vertical axis depicts confidence. A reading 661 represents the confidence level CL of the peaks 651. On the vertical axis, CLmin is a minimum confidence level which the reading should exceed. CL is the confidence level of the reading 612. Since CL>CLmin, the active state is terminated before another heart rate reading is obtained, in one approach.

FIG. 7A depicts a plot of breathing rate in different predetermined phases of sleep, consistent with step 340 of FIG. 3A. The horizontal axis depicts time and the vertical axis depicts a breathing rate, e.g., breathes per minute. The breathing rate can be determined by the motion sensor as mentioned. The user is awake from t0-t1 and t6-t7. A sleep-related activity can include onset of sleep from t1-t2, non-REM sleep from t2-t3, REM sleep from t3-t4, non-REM sleep from t4-t5, and waking up from t5-t6. The motion sensor can be used to provide as an initial indication that the user is in a sleep-related activity. This indication could be based on other factors as well such as information from the ambient light sensor, the time of day, and skin temperature, which decreases while a user is sleeping. If a sleeping activity of interest is indicated by the motion data, the heart rate sensor can be activated to obtain readings. One purpose of the heart rate readings is to associate them with the sleep-related activity to assess the quality of the user's sleep. The heart rate readings can also be used to confirm the identification of the sleep-related activity based on the motion data. If the heart rate readings do not confirm the identification of the sleep-related activity based on the motion data, one approach is assume the motion-based identification of the sleep-related activity is incorrect. Or, additional motion data and/or heart rate readings may be gathered to confirm the identification of the sleep-related activity.

BRth is a threshold breathing rate. The current breathing rate being above the threshold can be an indication that the user is awake, while the current breathing rate being below the threshold can be an indication that the user is sleeping or otherwise in a sleep-related activity.

FIG. 7B depicts a plot of a slope of the breathing rate of FIG. 7A. The horizontal axis depicts time, aligned with the horizontal axis of FIG. 7A, and the vertical axis depicts the slope, e.g., rate of change, of the breathing rate. A slope of zero indicates a constant breathing rate. A range of +/− delta (e.g., +/−5%) is around the slope of zero. A slope within this range represents a substantially constant breathing rate. A slope above +delta represents an increasing breathing rate and a slope below −delta represents a decreasing breathing rate.

FIG. 7C depicts a plot of heart rate in different predetermined phases of sleep, consistent with FIG. 7A. The heart rate generally tracks the breathing rate so that it is steady, increasing or decreasing when the breathing rate is steady, increasing or decreasing, respectively. HRTh is a threshold heart rate. The current heart rate being above the threshold can be an indication that the user is awake, while the current heart rate being below the threshold can be an indication that the user is sleeping or otherwise in a sleep-related activity. A slope of the heart rate (not shown) can also be determined. A slope of zero indicates a constant heart rate. A range of +/− delta (e.g., +/−5%) can be around the slope of zero. A slope within this range represents a substantially constant heart rate. A slope above +delta represents an increasing heart rate and a slope below −delta represents a decreasing heart rate.

As mentioned, the user is awake from t0-t1 and t6-t7. This can be determined initially based on the breathing rate being above BRTh and subsequently confirmed by the heart rate being above HRTh.

The motion sensor can be used as an initial indication that the user is in a sleep-related activity. The heart rate can be used as a confirmation of the sleep-related activity and for use in evaluating the quality of the users sleep.

In FIG. 7B, from t0-t1, the slope of the breathing rate alternates between positive and negative values. In one approach, a measure of variability is proportional to the number of zero crossings (changes from positive to negative or from negative to positive) in the slope in a given time period. An example measure is three crossings per minute. Thus, a relatively higher number of zero crossing is associated with a relatively higher variability. Other measures of variability can be used as well. One example uses a range of the breathing rate in a time period, such that a relatively higher range is associated with a relatively higher variability. The breathing rate from t0-t1 has a medium variability. This period is associated with the user being awake. In some cases, the breathing rate may not be discernable when the user is awake because of movements other than breathing that the user makes. However, this does not prevent the breathing rate from being used to detect the sleep-related activities. The detection of the awake period is confirmed by the heart rate being above HRTh in FIG. 7C.

From t1-t2, the slope of the breathing rate remains negative and below −delta so that the breathing rate is steadily decreasing. Also, there are no zero crossings so that the variability is low. This period is associated with the onset of sleep, as is confirmed by the decreasing heart rate in FIG. 7C (e.g., the heart rate decreases below HRTh).

From t2-t3, the slope of the breathing rate remains at zero so that the breathing rate is constant and the variability is low. This period is associated with non-REM sleep, as is confirmed by the constant heart rate which is above HRTh.

From t3-t4, the slope of the breathing rate alternates between positive and negative values. There are several zero crossings so that the variability is high. This period is associated with the REM sleep, as is confirmed by the elevated heart rate (the heart rate being above HRTh).

From t5-t6, the slope of the breathing rate remains at zero so that the breathing rate is constant and the variability is low. This period is associated with non-REM sleep, as is confirmed by the constant heart rate which is below HRTh.

From t6-t7, the slope of the breathing rate remains positive and above +delta so that the breathing rate is steadily increasing. Also, there are no zero crossings so that the variability is low. This period is associated with the user waking up, as is confirmed by the increasing heart rate (e.g., the heart rate increases above HRTh).

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto.

What is claimed is:

1. A method for monitoring a heart rate of a user, comprising:
obtaining motion data from a motion sensor worn by the user which indicates the user is not engaging in a threshold level of activity;
keeping a heart rate sensor worn by the user in a schedule-based mode in response to the motion data indicating the user is not engaging in the threshold level of activity, the heart rate sensor in the schedule-based mode obtains a heart rate of the user at scheduled times which are not based on the motion data and does not obtain a heart rate of the user at other times;
obtaining motion data from the motion sensor which indicates the user is engaging in the threshold level of activity;
in response to the motion data which indicates the user is engaging in the threshold level of activity, providing the heart rate sensor in an alternating mode instead of in the schedule-based mode, the heart rate sensor in the alternating mode repeatedly alternates between an active state in which the heart rate sensor obtains the heart rate of the user and an inactive state in which the heart rate sensor does not obtain the heart rate of the user;
obtaining motion data from the motion sensor during the alternating mode; and
setting a duration of the inactive state based on the motion data which is obtained from the motion sensor during the alternating mode.

2. The method of claim 1, wherein:
the duration of the inactive state varies in inverse proportion to an amount of motion which is indicated by the motion data which is obtained from during the alternating mode.

3. The method of claim 1, wherein:
the motion data during the alternating mode comprises an activity count; and
the duration of the inactive state is inversely proportional to the activity count.

4. The method of claim 1, further comprising:
determining a type of activity of the user based on the motion data obtained during the alternating mode;
setting the duration of the inactive state to be relatively longer when the type of activity is relatively less vigorous; and
setting the duration of the inactive state to be relatively shorter when the type of activity is relatively more vigorous.

5. The method of claim 1, wherein:
the motion data which indicates the user is engaging in the threshold level of activity indicates that the user is engaged in a particular type of activity.

6. The method of claim 1, wherein:
the motion data which indicates the user is engaging in the threshold level of activity comprises an activity count by the user which exceeds a threshold.

7. The method of claim 1, further comprising:
setting the duration of the inactive state based on values of the heart rate obtained during the alternating mode, the duration of the inactive state is inversely proportional to values of the heart rate obtained during the alternating mode.

8. The method of claim 1, wherein:
each active state in the alternating mode extends over a time period which is sufficient for the heart rate of the user to be obtained with a confidence level which exceeds a minimum confidence level and is concluded in response to the heart rate of the user being obtained with the confidence level which exceeds the minimum confidence level, or over a maximum allowable duration if the heart rate of the user cannot be obtained with the confidence level which exceeds the minimum confidence level before an end of the maximum allowable duration.

9. The method of claim 1, wherein:
the heart rate sensor emits light during the active state and does not emit light during the inactive state.

10. A method for monitoring a heart rate of a user, comprising:
determining that motion data from a motion sensor worn by a user is not consistent with the user engaging in vigorous exercising in a first time period;
in response to the determining that the motion data in the first time period is not consistent with the user engaging in vigorous exercising, providing a heart rate sensor of the user in an alternating mode in the first time period, the heart rate sensor in the alternating mode repeatedly alternates between an active state in which the heart rate sensor obtains a heart rate of the user and an inactive state in which the heart rate sensor does not obtain the heart rate of the user;
determining that motion data from the motion sensor is consistent with the user engaging in vigorous exercising in a second time period directly after the first time period;
in response to the determining that the motion data in the second time period is consistent with the user engaging in vigorous exercising, providing the heart rate sensor continuously in the active state in the second period;
determining that motion data from the motion sensor is not consistent with the user engaging in the vigorous exercising in a third time period directly after the second time period;
in response to the determining that the motion data in the third time period is not consistent with the user engaging in vigorous exercising, keeping the heart rate sensor continuously in the active state until the heart rate is determined to have to fallen to within a range of a resting heart rate of the user; and
storing, as a heart rate recovery time of the user, a time elapsed between a start of the third time period, when the motion data in the third time period initially indicates the user has terminated the vigorous exercising, and a time at which the heart rate is determined to have to fallen to within the range of the resting heart rate of the user.

11. The method of claim 10, wherein:
the active state extends over multiple heart beat periods and the inactive state extends over multiple heart beat periods.

12. The method of claim 10, further comprising:
in the third time period, in response to the determining that the heart rate has fallen to within the range of the resting heart rate of the user, providing the heart rate sensor in the alternating mode.

13. The method of claim 10, wherein:
the motion data in the second time period comprises an activity count by the user; and
the motion data in the second time period is consistent with the user engaging in the vigorous exercising when the activity count exceeds a threshold count.

14. The method of claim 10, wherein:
the determining that the motion data in the second time period is consistent with the user engaging in vigorous exercising is based on the motion data in the second time period correlating with a signature of a vigorous exercise.

15. The method of claim 10, further comprising:
determining that the heart rate in the second time period is consistent with the user engaging in vigorous exercising based on the heart rate in the second time period exceeding a threshold for detection of vigorous exercise, which is above the range of the resting heart rate by at least a specified amount, the providing the heart rate sensor continuously in the active state in the second period is also responsive to the determining that the heart rate in the second time period is consistent with the user engaging in vigorous exercising.

16. The method of claim 10, further comprising:
determining that the heart rate in the first time period is not consistent with the user engaging in vigorous exercising based on the heart rate in the first time period remaining below a threshold, the providing the heart rate sensor in the alternating mode in the first time period
is also responsive to the determining that the heart rate in the first time period is not consistent with the user engaging in vigorous exercising.

17. A monitor, comprising:
a heart rate sensor worn by a user;
a motion sensor worn by the user; and
a processor, the processor:
obtains motion data from the motion sensor which indicates the user is not in a predetermined phase of sleep;
keeps the heart rate sensor in a schedule-based mode in response to the motion data indicating the user is not in the predetermined phase of sleep, the heart rate sensor in the schedule-based mode obtains a heart rate of the user at scheduled times which are not based on the motion data and does not obtain a heart rate of the user at other times;
obtains motion data from the motion sensor which indicates the user is in the predetermined phase of sleep; and
in response to the motion data which indicates the user is in the predetermined phase of sleep, providing the heart rate sensor in an active state in which the heart rate sensor obtains values of the heart rate of the user at times which are outside of the scheduled times and identifying the values of the heart rate which are obtained while the heart rate sensor is in the active state as being associated with the predetermined phase of sleep.

18. The monitor of claim 17, wherein:
the motion data which indicates the user is not in the predetermined phase of sleep indicates a steady breathing rate of the user; and
the processor confirms that the user is not engaged in the predetermined phase of sleep when the values of the heart rate which are obtained while the heart rate sensor is in the active state indicate a steady heart rate of the user.

19. The monitor of claim 17, wherein:
the predetermined phase of sleep comprises an onset of sleep;
the motion data which indicates the user is in the predetermined phase of sleep indicates a steady breathing rate of the user which is below a threshold; and
the processor confirms that the user is in the predetermined phase of sleep when the values of the heart rate which are obtained while the heart rate sensor is in the active state indicate a decreasing heart rate of the user which is below a threshold.

20. The monitor of claim 17, wherein:
the predetermined phase of sleep comprises a rapid eye movement sleep;
the motion data which indicates the user is in the predetermined phase of sleep indicates an increasing breathing rate of the user which is above a threshold; and
the processor confirms that the user is in the predetermined phase of sleep when the values of the heart rate which are obtained while the heart rate sensor is in the active state indicate an increasing heart rate of the user which is above a threshold.

21. The monitor of claim 17, wherein:
the predetermined phase of sleep comprises a non-rapid eye movement sleep;
the motion data which indicates the user is in the predetermined phase of sleep indicates a steady breathing rate of the user which is below a threshold; and
the processor confirms that the user is in the predetermined phase of sleep when the values of the heart rate which are obtained while the heart rate sensor is in the active state indicate a steady heart rate of the user which is below a threshold.

22. The monitor of claim 17, wherein:
the predetermined phase of sleep comprises the user waking up from sleep;
the motion data which indicates the user is in the predetermined phase of sleep indicates a change in posture of the user from lying to sitting or standing; and
the processor confirms that the user is in the predetermined phase of sleep when the values of the heart rate which are obtained while the heart rate sensor is in the active state indicate an increasing heart rate of the user which is above a threshold.

23. The monitor of claim 17, wherein:
a resting heart rate of the user is determined in response to the motion data which indicates the user is waking up from sleep.
